(19) United States
(12) Patent Application Publication
(10) Pub. No.: US 2024/0029859 A1
(43) Pub. Date: Jan. 25, 2024

(54) HEALTH MANAGEMENT SUPPORT SYSTEM, ITS CONTROL METHOD, AND CONTROL PROGRAM

(71) Applicant: TOYOTA JIDOSHA KABUSHIKI KAISHA, Aichi-ken (JP)

(72) Inventors: Issei NAKASHIMA, Tokyo-to (JP); Toru MIYAGAWA, Seto-shi (JP); Hideo HASEGAWA, Nagoya-shi (JP); Yusuke KINOSHITA, Tokyo-to (JP); Hiroki SATO, Tokyo-to (JP); Hiroaki KAMEI, Taito-ku, Tokyo-to (JP); Maika HASHIMOTO, Urayasu-shi (JP); Manabu YAMAMOTO, Toyota-shi (JP)

(21) Appl. No.: 18/356,160

(22) Filed: Jul. 20, 2023

(30) Foreign Application Priority Data
Jul. 25, 2022 (JP) 2022-117834

(51) Int. Cl. G16H 20/60; G16H 20/30; G16H 50/30

(52) U.S. Cl. CPC G16H 20/60; G16H 20/30; G16H 50/30

(57) ABSTRACT

A health management support system includes: a first acquisition unit configured to acquire information about a user, including a health condition of the user; a second acquisition unit configured to acquire information about at least one purchase-candidate food; an estimation unit configured to estimate an effect on the health condition of the user that is caused when the user ingests the at least one purchase-candidate food; a calculation unit configured to calculate a cost that is incurred to alleviate the effect on the health condition of the user estimated by the estimation unit; and an output unit configured to output the effect on the health condition of the user estimated by the estimation unit and the cost calculated by the calculation unit.

### CROSS REFERENCE TO RELATED APPLICATIONS

This application is based upon and claims the benefit of priority from Japanese patent application No. 2022-117834, filed on Jul. 25, 2022, the disclosure of which is incorporated herein in its entirety by reference.

### BACKGROUND

The present disclosure relates to a health management support system, its control method, and a control program.

Japanese Unexamined Patent Application Publication No. 2010-157123 discloses a health guidance support program for making a computer or the like function as data receiving means for receiving information about the health of a user, and future prediction means for showing both a prediction in the future when the life of the user is improved and that when the life of the user is not improved based on the information about the health of the user received by the data receiving means. It should be noted that Japanese Unexamined Patent Application Publication No. 2010-157123 discloses that the prediction in the future is a prediction about at least one item selected from a group consisting of a body shape, an age that a user seems to be based on his/her health condition, and a medical expense.

### SUMMARY

However, in Japanese Unexamined Patent Application Publication No. 2010-157123, there is a problem that it does not show an economic risk caused by a disease that may occur when a purchase-candidate food is purchased.

The present disclosure has been made in view of the above-described circumstances, and an object thereof is to provide a health management support system, its control method, and a control program capable of showing, to a user, an economic risk caused by a disease that the user may suffer from when he/she purchases a purchase-candidate food, to thereby urge the user to purchase foods suitable for him/her.

A health management support system according to an aspect of the present disclosure includes: a first acquisition unit configured to acquire information about a user, including a health condition of the user; a second acquisition unit configured to acquire information about at least one purchase-candidate food; an estimation unit configured to estimate an effect on the health condition of the user that is caused when the user ingests the at least one purchase-candidate food; a calculation unit configured to calculate a cost that is incurred to alleviate the effect on the health condition of the user estimated by the estimation unit; and an output unit configured to output the effect on the health condition of the user estimated by the estimation unit and the cost calculated by the calculation unit. The above-described health management support system can show, to a user, an economic risk caused by a disease that the user may suffer from when he/she purchases a purchase-candidate food. As a result, the user can purchase appropriate foods while taking the economic risk into consideration.

The information about the user may include information about at least one of an age, a gender, a height, and a weight of the user. The accuracy of the estimation of the effect on the health condition of the user that is caused when he/she purchases a purchase-candidate food can be improved by taking the gender, the age, and the like of the user, in addition to his/her health condition, into consideration.

The information about the user may include information about at least either of a food that the user is recommended to refrain from ingesting and a food that the user is prohibited to ingest. In this way, it is possible to prevent the user from unintentionally purchasing a food that the user is recommended to refrain from ingesting and a food that the user is prohibited to ingest.

The information about the at least one purchase-candidate food may include information about a type of the at least one purchase-candidate food and a nutrient contained therein.

The estimation unit may infer a disease that the user may suffer from as the effect on the health condition of the user.

The estimation unit may extract, from among food purchase histories and changes in health conditions of a plurality of subjects in the past registered in a first database, a food purchase history and a change in health condition of at least one subject whose health condition has been determined to be similar to that of the user, and the estimation unit may estimate the effect on the health condition of the user by referring to the extracted food purchase history and the change in the health condition of the subject.

The calculation unit may extract, from among diseases that a plurality of past subjects suffered from respectively, and costs that were incurred to treat the diseases, registered in a second database, a cost that was incurred to treat a disease of at least one of the subjects who suffered from the disease that the user may suffer from, inferred (estimated) as the effect on the health condition of the user by the estimation unit, and the calculation unit may calculate, by referring to the extracted cost, a cost that is incurred to treat the disease that the user may suffer from, inferred by the estimation unit.

The health management support system may further include a selection unit configured to select, as an alternative food, a substitute food for at least one of the at least one purchase-candidate food when the effect on the health condition of the user estimated by the estimation unit exceeds a predetermined degree of effect, in which the output unit may further output information about the alternative food.

A method for controlling a health management support system according to an aspect of the present disclosure includes: acquiring information about a user, including a health condition of the user; acquiring information about at least one purchase-candidate food; estimating an effect on the health condition of the user that is caused when the user ingests the at least one purchase-candidate food; calculating a cost that is incurred to alleviate the estimated effect on the health condition of the user; and outputting the estimated effect on the health condition of the user and the calculated cost. By the above-described method for controlling a health management support system, it is possible to show, to a user, an economic risk caused by a disease that the user may suffer from when he/she purchases a purchase-candidate food. As a result, the user can purchase appropriate foods while taking the economic risk into consideration.

A control program according to an aspect of the present disclosure causes a computer to perform: a process for acquiring information about a user, including a health condition of the user; a process for acquiring information about at least one purchase-candidate food; a process for estimating an effect on the health condition of the user that is caused when the user ingests the at least one purchase-candidate food; a process for calculating a cost that is incurred to alleviate the estimated effect on the health condition of the user; and a process for outputting the estimated effect on the health condition of the user and the calculated cost. By the above-described control program unit, it is possible to show, to a user, an economic risk caused by a disease that the user may suffer from when he/she purchases a purchase-candidate food. As a result, the user can purchase appropriate foods while taking the economic risk into consideration.

According to the present disclosure, it is possible to provide a health management support system, its control method, and a control program capable of showing, to a user, an economic risk caused by a disease that the user may suffer from when he/she purchases a purchase-candidate food, to thereby urge the user to purchase foods suitable for him/her.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a health management support system according to a first embodiment;

FIG. 2 is a block diagram showing an example of a specific configuration of a health management support apparatus provided in the health management support system shown in FIG. 1;

FIG. 3 is a block diagram showing a modified example of the health management support system shown in FIG. 1;

FIG. 4 is a block diagram showing an example of a specific configuration of a health management support apparatus provided in the health management support system shown in FIG. 3;

FIG. 5 is a flowchart showing operations performed by the health management support system shown in FIG. 1; and

FIG. 6 shows an example of contents displayed on a monitor of a user terminal.

### DESCRIPTION OF EMBODIMENTS

The present disclosure will be described hereinafter through embodiments of the disclosure, but the invention according to the claims is not limited to the below- shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### <First Embodiment>

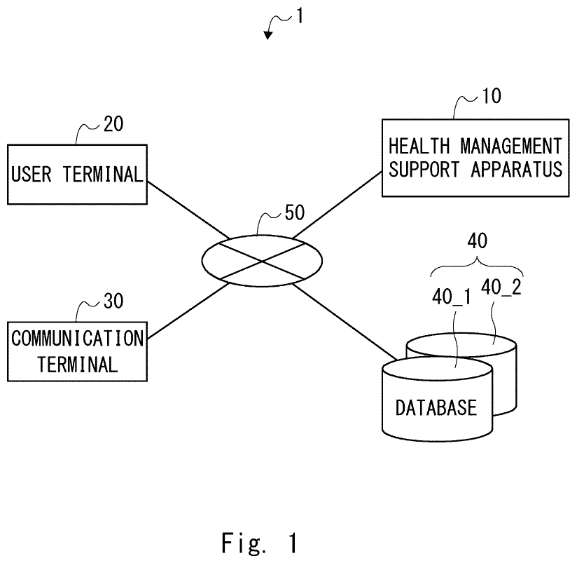

FIG. 1 is a block diagram showing an example of a configuration of a health management support system 1 according to a first embodiment. In health management support system 1, an economic risk caused by a disease that the user may suffer from when he/she purchases a purchase-candidate food is shown to a user, so that the user can purchase appropriate foods while taking the economic risk into consideration. That is, the health management support system 1 can urge the user to purchase foods suitable for him/her. Details of the health management support system 1 or the like will be described hereinafter.

As shown in FIG. 1, the health management support system 1 includes a health management support apparatus 10, a user terminal 20, a communication terminal 30, a database 40, and a network 50. The database 40 includes databases 40_1 and 40_2. The health management support apparatus 10 can also be referred to as a health management support system. The health management support apparatus 10, the user terminal 20, the communication terminal 30, and the database 40 are configured so that they can communicate with each other through the wired or wireless network 50.

The user terminal 20 is a terminal owned by or temporarily assigned to a user U1 and capable of performing communication such as a PC (Personal Computer) terminal, a mobile terminal such as a smartphone or a tablet-type terminal, or a dedicated communication terminal provided for the health management support system. For example, the user U1 enters information about the user U1 into the user terminal 20 by operating an operation screen, a keyboard, or the like of the user terminal 20. The user terminal 20 receives the information about the user U1 entered by the operation performed by the user U1, and transmits the entered information to the health management support apparatus 10 through the network 50.

Note that the information about the user U1 includes at least information about the health condition of the user U1. The information about the health condition of the user U1 includes, for example, information as to whether or not the health condition of the user U1 is good and, when the user U1 is ill, information about the development of the disease. Note that a result of a health examination of the user U1 may be used as the information about the health condition of the user U1.

Further, the information about the user U1 may include at least one of the age, the gender, the height, and the weight of the user U1. In this way, it is possible to improve the accuracy of the estimation of the effect on the health condition of the user U1 that is caused when the user U1 ingests a purchase-candidate food (e.g., a food of which the user U1 is considering purchase), made by the health management support apparatus 10 (which will be described later).

Further, the information about the user U1 may include information about at least one of foods that the user U1 is recommended to refrain from ingesting and foods that the user U1 is prohibited to ingest. As the foods and the like that the user U1 is recommended to refrain from ingesting, foods that are roughly classified, such as being classified into fish, meat, vegetables, and the like, may be designated, or foods that are finely classified, such as being classified into pike, salmon, eel, and the like, may be designated. More specifically, as the foods and the like that the user U1 is recommended to refrain from ingesting, for example, eggs, milk, wheat, or buckwheat, which may cause food allergies, may be designated. Alternatively or additionally, fatty foods such as fatty tuna or honey, which infants are recommended to refrain from ingesting, may be designated. In this way, it is possible to improve the accuracy of the estimation of the effect on the health condition of the user U1 that is caused when the user U1 ingests a purchase-candidate food, made by the health management support apparatus 10 (which will be described later). Further, it is possible to prevent the user U1 from unintentionally purchasing a food that the user U1 is prohibited to ingest.

Note that although an example case where the user terminal 20 receives information about the user U1 and transmits the received information to the health management support apparatus 10 through the network 50 has been described in this embodiment, the present disclosure is not limited to this example. For example, a communication terminal (not shown) installed in a hospital may receive a result or the like of a health examination or a medical examination of the user U1 and transmit the received result or the like to the health management support apparatus 10 through the network 50. Alternatively, a wearable terminal (not shown) worn by the user U1 may transmit vital data such as a heart rate and a pulse rate of the user U1 to the health management support apparatus 10 through the network 50.

The communication terminal 30 is, for example, a terminal capable of performing communication, installed in each shopping basket of a store where foods are sold, such as a PC terminal, a mobile terminal such as a tablet-type terminal, or a dedicated communication terminal provided for the health management support system. The communication terminal 30 reads, for example, information about a purchase-candidate food put in a shopping basket by the user U1 from a label attached to the food or a result of an analysis of a photographed image of the food, and transmits the read information to the health management support apparatus 10 through the network 50.

Note that although an example case where the communication terminal 30 is installed in each shopping basket of one store has been described in this embodiment, the present disclosure is not limited to this example. The communication terminal 30 may be installed in each shopping basket in a plurality of stores. Further, instead of the communication terminal 30, the user terminal 20 may read information about a purchase-candidate food and transmit the read information to the health management support apparatus 10 through the network 50.

The health management support apparatus 10 estimates the effect on the health condition of the user U1 that is caused when the user U1 ingests a purchase-candidate food, calculates a cost that is incurred to alleviate the estimated effect on the health condition of the user U1 (e.g., to treat the disease that the user U1 suffers from because of the ingestion of a purchase-candidate food), and outputs the result of the estimation and the result of the calculation.

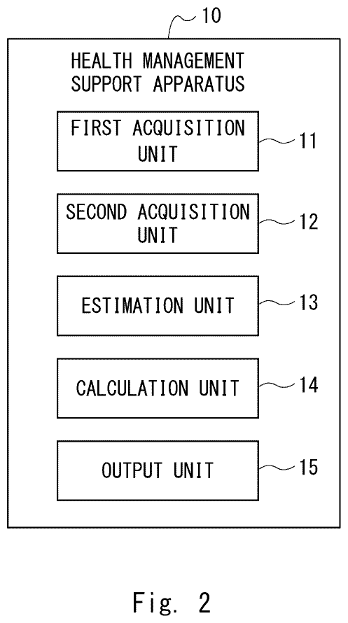

FIG. 2 is a block diagram showing an example of a configuration of the health management support apparatus 10. As shown in FIG. 2, the health management support apparatus 10 includes at least a first acquisition unit 11, a second acquisition unit 12, an estimation unit 13, a calculation unit 14, and an output unit 15.

The first acquisition unit 11 acquires information about the user U1 including his/her health condition, transmitted from the user terminal 20 or the like.

The second acquisition unit 12 acquires information about purchase-candidate foods of the user U1 (e.g., foods of which the user U1 is considering purchase), transmitted from the communication terminal 30 or the like installed in a shopping basket or the like of the store. Note that the information about the purchase-candidate food may include information about a type of the purchase-candidate food and a nutrient(s) contained therein.

The estimation unit 13 estimates the effect on the health condition of the user U1 that is caused when the user U1 ingests a purchase-candidate food. For example, the estimation unit 13 infers a disease that the user U1 may suffer from as the effect on the health condition of the user U1.

Specifically, for example, when the purchase-candidate food is an allergen food such as an egg, the estimation unit 13 infers an occurrence of a food allergy as an effect on the health condition of the user U1. Further, for example, when the purchase-candidate food is a fatty food, the estimation unit 13 infers an onset of a metabolic syndrome as an effect on the health condition of the user U1. Further, for example, when the purchase-candidate food is a food containing a large amount of a carcinogenic substance(s), the estimation unit 13 infers an onset of a cancer in a predetermined part of the body as an effect on the health condition of the user U1. Further, for example, when the purchase-candidate food is a food containing a large amount of sugar, the estimation unit 13 infers, for example, an onset of diabetes or an onset of osteoporosis as an effect on the health condition of the user U1. Alternatively, when a plurality of purchase-candidate foods are ingested and an amount of a predetermined component(s) ingested is excessive, the estimation unit 13 may infer a disease that the user U1 may suffer from as he/she ingests an excessive amount of the predetermined component(s).

Note that the estimation by the estimation unit 13 is not limited to the estimation of the effect on the health condition of the user U1 for each purchase-candidate food. For example, the estimation unit 13 may estimate the effect on the health condition of the user U1 that is caused by the accumulation of a plurality of foods purchased in a predetermined period in the past and a purchase-candidate food(s).

Further, the estimation unit 13 may also estimate, in addition to a disease that the user U1 may suffer from, a probability of an occurrence of that disease as the effect on the health condition of the user U1. Note that the effect on the health condition of the user U1 that is caused when the user U1 ingests a purchase-candidate food could change depending on, for example, the current health condition of the user U1, and/or the age, the gender, the height, and the weight of the user U1. Accordingly, the larger the amount of information about the user U1 acquired by the first acquisition unit 11 is, the more the estimation unit 13 can improve the accuracy of the estimation.

The estimation unit 13 will be described in a more detailed manner. For example, in the database 40_1, a food purchase history and changes in the health condition of each of a plurality of the users (subjects) in the past are registered. The estimation unit 13 extracts, from the database 40_1, a food purchase history and changes in the health condition of at least one subject whose health condition has been determined to be similar to that of the current user U1. Then, the estimation unit 13 estimates, by referring to the food purchase history and the changes in the health condition of the extracted subject, the effect on the health condition of the user U1 that is caused when the user U1 ingests a purchase-candidate food.

Note that in the database 40_1, in addition to the food purchase history and the changes in the health condition of each of the plurality of subjects in the past, at least one of the age, the gender, the height, and the weight of each of the plurality of subjects in the past may also be registered. In this way, the estimation unit 13 can easily narrow down the conditions, and thereby can improve the accuracy of the estimation.

The calculation unit 14 calculates a cost that is incurred to alleviate the effect on the health condition of the user U1 that is caused when the user U1 ingests a purchase-candidate food (e.g., to treat a disease that the user U1 may suffer from when he/she ingests a purchase-candidate food), estimated by the estimation unit 13. This cost includes at least a cost for treating the disease. Further, this cost may also include, for example, a transportation expense for a clinic or a hospital.

The calculation unit 14 will be described in a more detailed manner. For example, in the database 40_2, diseases that a plurality of past users (subjects) suffered from respectively, and costs that were incurred to treat the diseases are registered. The calculation unit 14 extracts, from the database 40_2, a cost that was incurred to treat a disease of at least one of the subjects who suffered from the disease that the user U1 may suffer from, inferred by the estimation unit 13.

Then, the calculation unit 14 calculates, by referring to the extracted cost, a cost that is incurred to treat the disease that the user U1 may suffer from, inferred by the estimation unit 13.

Note that in the database 40_2, in addition to the diseases that the plurality of past subjects suffered from respectively, and the costs that were incurred to treat the diseases, at least one of the age, the gender, the height, and the weight of each of the plurality of past subjects may also be registered. In this way, the calculation unit 14 can easily narrow down the conditions, and thereby can improve the accuracy of the calculation.

The output unit 15 outputs the result of the estimation by the estimation unit 13 and the result of the calculation by the calculation unit 14. Specifically, the output unit 15 outputs the effect on the health condition of the user U1 that is caused when the user U1 ingests a purchase-candidate food and the cost that is incurred to alleviate the effect on the health condition of the user U1. The information output from the output unit 15 is transferred, for example, to the user terminal 20 through the network 50, and displayed on the monitor of the user terminal 20. In this way, the user U1 can recognize an economic risk caused by a disease that the user may suffer from when he/she ingests a purchase-candidate food.

Note that the output unit 15 may be configured to output, when the purchase-candidate food is at least one of foods that the user U1 is recommended to refrain from ingesting and foods that the user U1 is prohibited to ingest, information indicating as such. This information output from the output unit 15 is transferred, for example, to the user terminal 20 through the network 50 and is output from a speaker(s) of the user terminal 20 in the form of voice. In this way, it is possible to prevent the user U1 from unintentionally purchasing a food that the user U1 is prohibited to ingest.

As described above, in the health management support system 1 according to this embodiment, an economic risk caused by a disease that the user U1 may suffer from when he/she ingests a purchase-candidate food is shown to the user U1, so that the user U1 can purchase appropriate foods while taking the economic risk into consideration. That is, the health management support system 1 can urge the user U1 to purchase foods suitable for him/her.

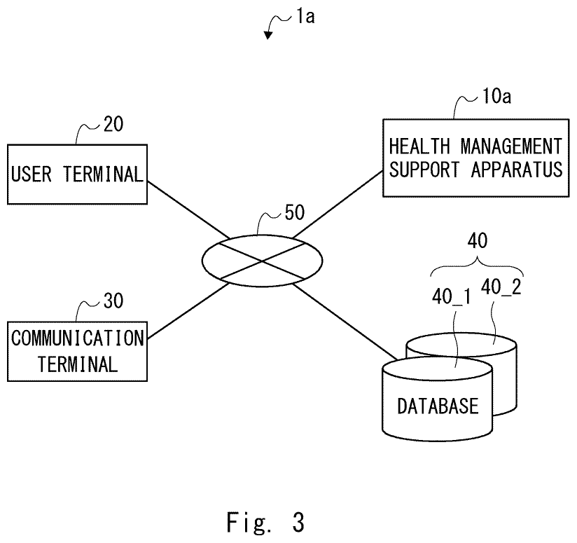

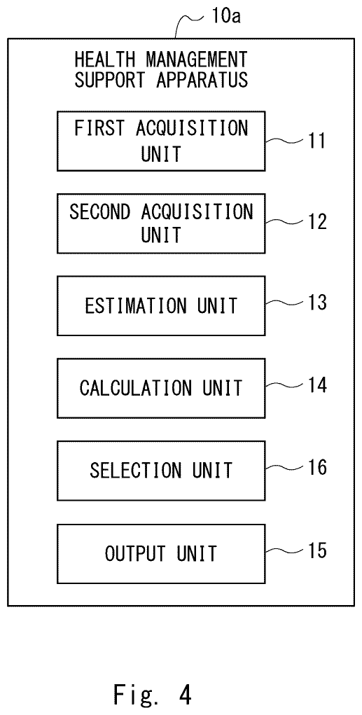

Note that the health management support apparatus 10 may be configured to, when the effect on the health condition of the user U1 that is caused when the user U1 ingests at least one purchase-candidate food exceeds a predetermined degree of effect, propose, as an alternative food, a substitute food for at least one of the at least one purchase-candidate food. This feature will be briefly described hereinafter with reference to FIGS. 3 and 4.

FIG. 3 is a block diagram showing a modified example of the health management support system 1 as a health management support system 1a. The health management support system 1a includes a health management support apparatus 10a in place of the health management support apparatus 10. FIG. 4 is a block diagram showing an example of a specific configuration of the health management support apparatus 10a. The health management support apparatus further includes a selection unit 16 as compared with the health management support apparatus 10.

When the effect on the health condition of the user U1 that is caused when he/she ingests at least one purchase-candidate food exceeds a predetermined degree of effect, the selection unit 16 selects, as an alternative food, a substitute food for at least one of the at least one purchase candidate. For example, when a disease that the user U1 may suffer from when he/she ingests a purchase-candidate food is serious or when the probability of the onset of a disease is very high, the selection unit 16 selects, as an alternative food, a substitute food for the purchase-candidate food. Then, the output unit 15 also outputs, in addition to the result of the estimation by the estimation unit 13 and the result of the calculation by the calculation unit 14, information about the substitute food. The rest of the configuration of the health management support system 1a is similar to that of the health management support system 1, and therefore the description thereof is omitted.

### (Operation of Health Management Support System 1)

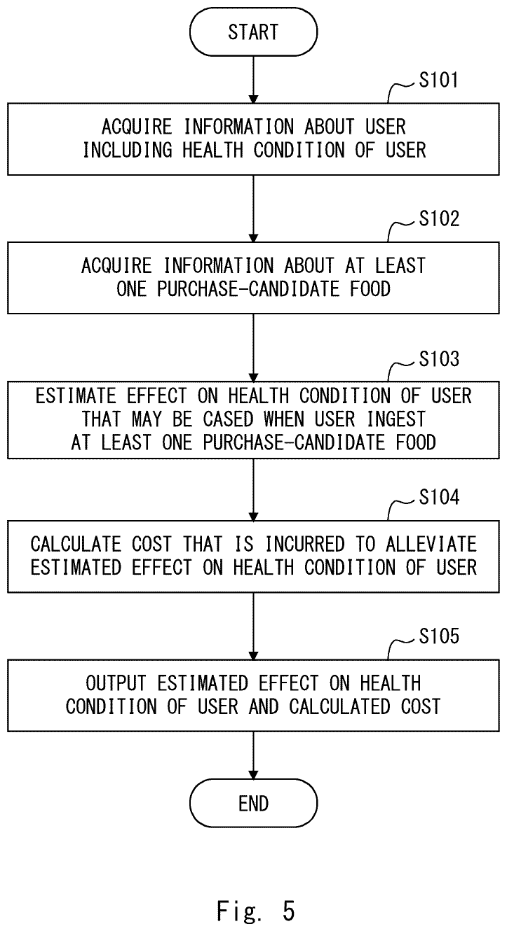

Next, operations performed by the health management support system 1 will be described with reference to FIG. 5. FIG. 5 is a flowchart showing operations performed by the health management support system 1.

Firstly, the user terminal 20, which is a smartphone or the like, receives information about a user U1 including the health condition of the user U1, entered by an operation performed by the user U1, and transmits the received information to the health management support apparatus 10 through the network 50. Note that as already described above, the apparatus or the like that receives and transmits information about the user U1 is not limited to the user terminal 20. That is a communication terminal installed in a hospital or a wearable terminal may receive information about the user U1 and transmit the received information to the health management support apparatus 10 through the network 50.

Further, the communication terminal 30 installed in a shopping basket of a store receives information about a purchase-candidate food that the user U1 has put in the shopping basket, and transmits the received information to the health management support apparatus 10 through the network 50. Note that as already described above, the apparatus or the like that receives and transmits information about a purchase-candidate food is not limited to the communication terminal 30. That is, the user terminal 20 may receive information about a purchase-candidate food that the user U1 has put in the shopping basket, and transmit the received information to the health management support apparatus 10 through the network 50.

In the health management support apparatus 10, the first acquisition unit 11 acquires the information about the user U1 including the health condition of the user U1, transmitted from the user terminal 20 or the like (Step S101).

Further, in the health management support apparatus 10, the second acquisition unit 12 acquires the information about at least one purchase-candidate food, transmitted from the communication terminal 30 or the like installed in the shopping basket of the store (Step S102). In this example, the second acquisition unit 12 acquires information about foods F1 to F3 as information about at least one purchase-candidate food.

After that, in the health management support apparatus 10, the estimation unit 13 estimates the effect on the health condition of the user U1 that is caused when the user U1 ingests at least one purchase-candidate food (Step S103).

Specifically, firstly, the estimation unit 13 extracts, from the database 40_1, a food purchase history and changes in the health condition of at least one subject whose health condition has been determined to be similar to that of the current user U1. Then, the estimation unit 13 estimates, by referring to the food purchase history and the changes in the health condition of the extracted subject, the effect on the health condition of the user U1 that is caused when the user U1 ingests a purchase-candidate food.

For example, the estimation unit 13 infers an occurrence of a food allergy as an effect on the health condition of the user U1 that is caused when the user U1 ingests the purchase-candidate food Fl. Further, in this process, the estimation unit 13 estimates that the probability of the occurrence of the food allergy when the user U1 ingests the purchase-candidate food F1 is 25%. Further, the estimation unit 13 infers an onset of a metabolic syndrome as an effect on the health condition of the user U1 that is caused when the user U1 ingests the purchase-candidate foods F2 and F3. Further, in this process, the estimation unit 13 estimates that the probabilities of the occurrence of the metabolic syndrome when the user U1 ingests the purchase-candidate foods F2 and F3 are 10% and 5%, respectively.

Then, in the health management support apparatus 10, the calculation unit 14 calculates a cost that is incurred to alleviate the effect on the health condition of the user U1 that is caused when the user U1 ingests the purchase-candidate food (e.g., to treat a disease that the user U1 may suffer from when he/she ingests the purchase-candidate food), estimated by the estimation unit 13 (Step S104).

Specifically, firstly, the calculation unit 14 extracts, from the database 40_2, a cost that was incurred to treat a disease of at least one of the subjects who suffered from the disease that the user U1 may suffer from, inferred by the estimation unit 13. Then, the calculation unit 14 calculates, by referring to the extracted cost, a cost that is incurred to treat the disease that the user U1 may suffer from, inferred by the estimation unit 13.

For example, the calculation unit 14 calculates a cost that is incurred to treat the food allergy, which the user U1 may suffer from as he/she ingests the purchase-candidate food F1, to be about 50,000 yen. Further, the calculation unit 14 calculates a cost that is incurred to treat the metabolic syndrome, which the user U1 may suffer from as he/she ingests the purchase-candidate foods F2 and F3, to be about 30,000 yen.

After that, the output unit 15 outputs the result of the estimation by the estimation unit 13 and the result of the calculation by the calculation unit 14 (Step S105). Specifically, the output unit 15 outputs the effect on the health condition of the user U1 that is caused when the user U1 ingests a purchase-candidate food and the cost that is incurred to alleviate the effect on the health condition of the user U1. The information output from the output unit 15 is transferred, for example, to the user terminal 20 through the network 50, and displayed on the monitor of the user terminal 20. In this way, the user U1 can recognize an economic risk caused by a disease that the user may suffer from when he/she ingests a purchase-candidate food.

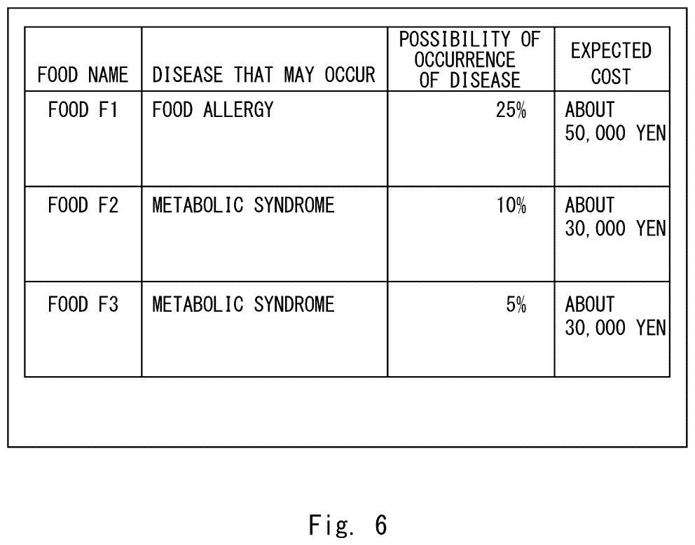

FIG. 6 shows an example of contents displayed on the monitor of the user terminal 20.

As shown in FIG. 6, on the monitor of the user terminal 20, the effects on the health condition of the user U1 that are caused when he/she ingests the purchase-candidate foods F1 to F3, respectively, and the expected costs that are incurred to alleviate the effects on the health condition of the user U1 are displayed. In the example shown in FIG. 6, as the effects on the health condition of the user U1, diseases that the user U1 may suffer from and the probabilities of occurrences (or onsets) thereof are shown.

As described above, in the health management support system 1 according to this embodiment, an economic risk caused by a disease that the user U1 may suffer from when he/she ingests a purchase-candidate food is shown to the user U1, so that the user U1 can purchase appropriate foods while taking the economic risk into consideration. That is, the health management support system 1 can urge the user U1 to purchase foods suitable for him/her.

Note that the present disclosure is not limited to the above-described embodiments, and they can be modified as appropriate without departing from the scope and spirit of the disclosure.

Further, in the present disclosure, some or all of the control processes performed in the health management support system 1 can be implemented by having a CPU (Central Processing Unit) execute a computer program.

In the aforementioned program includes instructions (or software codes) that, when loaded into a computer, cause the computer to perform at least one of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health management support system comprising:
a first acquisition unit configured to acquire information about a user, including a health condition of the user;
a second acquisition unit configured to acquire information about at least one purchase-candidate food;
an estimation unit configured to estimate an effect on the health condition of the user that is caused when the user ingests the at least one purchase-candidate food;
a calculation unit configured to calculate a cost that is incurred to alleviate the effect on the health condition of the user estimated by the estimation unit; and
an output unit configured to output the effect on the health condition of the user estimated by the estimation unit and the cost calculated by the calculation unit.

2. The health management support system according to claim 1, wherein the information about the user includes information about at least one of an age, a gender, a height, and a weight of the user.

3. The health management support system according to claim 1, wherein the information about the user includes information about at least either of a food that the user is recommended to refrain from ingesting and a food that the user is prohibited to ingest.

4. The health management support system according to claim 1, wherein the information about the at least one purchase-candidate food includes information about a type of the at least one purchase-candidate food and a nutrient contained therein.

5. The health management support system according to claim 1, wherein the estimation unit infers a disease that the user may suffer from as the effect on the health condition of the user.

6. The health management support system according to claim 1, wherein
the estimation unit extracts, from among food purchase histories and changes in health conditions of a plurality of subjects in the past registered in a first database, a food purchase history and a change in health condition of at least one subject whose health condition has been determined to be similar to that of the user, and
the estimation unit estimates the effect on the health condition of the user by referring to the extracted food purchase history and the change in the health condition of the subject.

7. The health management support system according to claim 1, wherein the calculation unit extracts, from among diseases that a plurality of past subjects suffered from respectively, and costs that were incurred to treat the diseases, registered in a second database, a cost that was incurred to treat a disease of at least one of the subjects who suffered from the disease that the user may suffer from, inferred as the effect on the health condition of the user by the estimation unit, and
the calculation unit calculates, by referring to the extracted cost, a cost that is incurred to treat the disease that the user may suffer from, inferred by the estimation unit.

8. The health management support system according to claim 1, further comprising a selection unit configured to select, as an alternative food, a substitute food for at least one of the at least one purchase-candidate food when the effect on the health condition of the user estimated by the estimation unit exceeds a predetermined degree of effect, wherein
the output unit further outputs information about the alternative food.

9. A method for controlling a health management support system, comprising:
acquiring information about a user, including a health condition of the user;
acquiring information about at least one purchase-candidate food;
estimating an effect on the health condition of the user that is caused when the user ingests the at least one purchase-candidate food;
calculating a cost that is incurred to alleviate the estimated effect on the health condition of the user; and
outputting the estimated effect on the health condition of the user and the calculated cost.

10. A non-transitory computer readable medium storing a control program for causing a computer to perform:
a process for acquiring information about a user, including a health condition of the user;
10 a process for acquiring information about at least one purchase-candidate food;
a process for estimating an effect on the health condition of the user that is caused when the user ingests the at least one purchase-candidate food;
a process for calculating a cost that is incurred to alleviate the estimated effect on the health condition of the user; and
a process for outputting the estimated effect on the health condition of the user and the calculated cost.
